(19) United States
(12) Patent Application Publication
(10) Pub. No.: US 2003/0134780 A1
(43) Pub. Date: Jul. 17, 2003

(54) Skin care compositions containing peptide copper complexes and retinol, retinol derivatives, or a mixture thereof

(75) Inventor: Leonard M. Patt, Seattle, WA (US)

Correspondence Address: SEED INTELLECTUAL PROPERTY LAW GROUP PLLC, 701 FIFTH AVE, SUITE 6300, SEATTLE, WA, 98104-7092, US

(73) Assignee: ProCyte Corporation, Redmond, WA

(21) Appl. No.: 10/264,363

(22) Filed: Oct. 4, 2002

Related U.S. Application Data
(60) Provisional application No. 60/327,640, filed on Oct. 5, 2001.

(51) Int. Cl. A61K 38/16; A61K 31/22; A61K 31/07

(52) U.S. Cl. 514/6; 514/546; 514/725

(57) ABSTRACT

Compositions, generally useful for preserving the skin and/or improving its health and appearance, comprise a peptide copper complex and retinol, a retinol derivative, or a mixture thereof. In another embodiment, the disclosed compositions further comprise additives, including emollients, sunscreen agents, skin protectants, skin conditioning agents, and/or humectants. Also disclosed is a method for treating skin to accomplish such preservation and/or improvement thereof, where the method comprises the step of topically applying a disclosed composition to an area of skin in need of such treatment.

### CROSS-REFERENCE TO RELATED APPLICATION

This application claims the benefit of U.S. Provisional Patent Application No. 60/327,640 filed Oct. 5, 2001, where this provisional application is incorporated herein by reference in its entirety.

### BACKGROUND OF THE INVENTION

1. Field of the Invention

The present invention generally relates to skin care compositions, and pharmaceutical and cosmetic preparations for skin, and more particularly, to compositions and preparations comprising peptide copper complexes and retinol, a retinol derivative, or a mixture thereof, as well as to methods for treating or preventing dermatological conditions related to photodamaged and aging skin.

2. Description of the Related Art

The use of chemical compositions to treat aged or photodamaged skin has been reported. For example the topical use of retinol (viatamin A) and retinol derivatives had been described for such treatment. More specifically, the retinol derivative, retonoic acid (present in Retin-A and Renova, Ortho Pharmaceuticals, Skillman, N.J.), has been shown to reduce the signs of photoaging (see J. Invest. Dermatology 104(4): 518-522, 1995). Retinoic acid compositions useful in skin treatment and cosmetic preparations have been disclosed, for example, in U.S. Pat. Nos. 5,955,109; 5,719,195; and 4,126,693.

Various compositions used for skin care applications comprising retinol, retinol derivatives, or mixtures thereof, in combination with other constituents have been described. For example, compositions containing fatty acid amides, in addition to retinol or retinyl ester, are described in U.S. Pat. No. 5,811,110. As another example, compositions containing geranyl geraniol, in addition to retinol or retinyl esters, are described in U.S. Pat. No. 5,756,109. As yet another example, compositions containing fatty hydroxyethyl imidazoline surfactants, in addition to retinol or retinol ester, are described in U.S. Pat. No. 5,738,858.

Also, copper is known to have many beneficial biological and cosmetic applications based on stimulating a variety of processes related to skin, such as collagen, elastin and glycosaminoglycan production (see, e.g., Maquart, F. X., Pickart, L., Laurent, M., Gillery, P., Monboisse, J. C., Borel, J. P., “Stimulation of Collagen Synthesis in Fibroblast Cultures by the Tripeptide-Copper Complex Glycyl-L-Histidyl-L-Lysine-Copper(2+),” FEBS Lett. 238(2): 343-346, 1988; Wegrowski, Y., Maquart, F. X. and Borel, J. P., “Stimulation of Sulfated Glycosaminoglycan Synthesis by the Tripeptide-Copper Complex Glycyl-L-Histidyl-L-Lysine-Copper(2+),” Life Sciences 51: 1049-1056, 1992; Maguart, F. X., Bellon, G., Chaqour, B., Wegrowski, J., Patt L. M., Trachy, R. E., Monboisse, J. C., Chastang, F., Birembaut, P., Gillery, P. and Borel, J. P., “In Vivo Stimulation of Connective Tissue Accumulation by the Tripeptide-Copper Complex Glycyl-L-Histidyl-L-Lysine-Copper(2+) in Rat Experimental Wounds,” J. Clin. Invest. 92: 2368-2376, 1993). The above-cited references are incorporated herein by reference in their entireties.

Copper salts alone are ineffective, or even inhibitory, for such applications. The copper must be delivered in a biologically acceptable form. As an example, when copper is complexed with a biologically acceptable carrier molecule, such as a peptide, it may then be effectively delivered to cells. More specifically, peptide copper complexes that have utility for wound healing and skin health when topically applied are described in U.S. Pat. Nos. 4,760,051: 4,665,054; 4,877,770; 5,135,913 and 5,348,943.

While chemical treatments, such as those described above, have demonstrated efficacy in the treatment of aged and photodamaged skin, there remains a need in the art for compositions demonstrating efficacy in such treatment greater than that achieved thus far, particularly when topically applied to skin in need of such treatment. The present invention fulfills these needs and provides further related advantages.

### BRIEF SUMMARY OF THE INVENTION

In one embodiment, the present invention provides compositions formed by combining retinol, at least one retinol derivative, or a mixture thereof, with at least one peptide copper complex. It has been surprisingly found that the ability of retinol or a retinol derivative to effect cellular proliferation and differentiation is increased when a peptide copper complex or peptide copper complex derivative is present. In addition, it has been surprisingly found that the effectiveness of a peptide copper complex in reducing the signs of photodamage or aging in the skin is enhanced when retinol or a retinol derivative is present. Thus, the present invention is based, at least in part, on the synergistic interaction between retinol or retinol derivatives and peptide copper complexes.

In another embodiment, there is disclosed such a composition where the retinol, the at least one retinol derivative, or mixture thereof, and the at least one peptide copper complex are encapsulated in liposomes or microsponges adapted to aid in delivery of the peptide copper complex, or to enhance the stability of the composition. In yet another embodiment, the components of the disclosed compositions are formulated in an instrument adapted to deliver the compounds via iontophoresis.

Additional embodiments of this invention are directed to the above compositions that further include an inert carrier or diluent, a sunscreen agent, a skin conditioning agent, a skin protectant, an emollient, a humectant, an excipient, a textural modifier, an emulsifying agent, a preserving agent, a thickening agent, or a mixture thereof. These compositions may be in the form of a solution, cream, gel, fluid cream or milk, lotion, or oil. Pharmaceutical and cosmetic preparations for skin, made from these compositions, are also disclosed.

The present invention is also directed to a method for treating skin by contacting the skin with an effective amount of a disclosed inventive composition or preparation. The effects of such treatment include conditioning and smoothening the skin, as well as reducing the signs of photodamage and aging of the skin, and reducing hyperpigmentation and wrinkling of the skin.

These and other aspects of this invention will be evident upon reference to the following detailed description of the invention.

### DETAILED DESCRIPTION OF THE INVENTION

As noted above, in one embodiment, disclosed is a composition formed by combining retinol, at least one retinol derivative, or a mixture thereof, and at least one peptide copper complex. As noted, it has been found that the disclosed composition has enhanced efficacy, to a surprising and unexpected extent, in the prevention and treatment of: photodamged skin, the appearance of fine lines and wrinkles, hyperpigmentation, age spots, and aged skin. The disclosed composition is also unexpectedly useful for increasing the flexibility of the stratum corneum, increasing the content of collagen and/or glycosaminoglycans in skin, increasing moisture in skin, decreasing transcutaneous water loss, and generally increasing the quality of skin. The disclosed composition also provides topical formulations effective in promoting a healthy scalp, and thereby useful in the prevention of hair loss, and as a treatment before and after hair transplant surgical procedures.

Retinol is also known as vitamin A and has the formula 3,7-dimethyl-9-(2,6,6-trimethyl-1-cyclohexen-1-yl)-2,4,6,8-nonatetraen-1-ol. Other terms that are used for retinol are axerophthol and vitamin A alcohol. In certain specific embodiments, the isomeric form of the retinol is all-trans-retinol; 1,3-cis-retinol; 3,4-didehydro-retinol; or 9-cis-retinol, respectively. In other embodiments of the above skin care composition, the retinol derivative is an ester of retinol selected from C₁-C₃₀ esters of retinol; C₂-C₂₀ esters of retinol; and C₂, C₃, and C₁₆ esters of retinol, respectively. More specifically, the ester of retinol may be retinyl palmitate, retinyl acetate and retinyl propionate. Other retinol derivatives that may be used are retinoic acid and retinyl aldehyde. The concentration of the retinol, retinol derivative, or mixture thereof, ranges from about 0.001% to about 10% in some embodiments; from about 0.01% to about 1% in other embodiments; and from about 0.01% to about 0.5% in yet other embodiments, by weight of the composition.

As used herein, the term “peptide copper complex” refers to a coordination compound comprising a peptide molecule and a copper ion non-covalently complexed therewith. The peptide molecule serves as the complexing agent by donating electrons to the copper ion to yield the non-covalent complex. The peptide molecule is a chain of two or more amino acid units covalently bonded together via amide linkages (for example, —CONH—), the formation of such linkages being accompanied by the elimination of water. The amino acid units are from amino acids that are naturally occurring or otherwise. Also, at least one amide linkage nitrogen atom may have covalently bonded thereto either a hydrogen atom or another moiety.

Generally, an amino acid consists of an amino group, a carboxyl group, a hydrogen atom, and an amino acid side-chain moiety—all bonded, in the case of an alpha-amino acid, to a single carbon atom that is referred to as an alpha-carbon. Compositions of the present invention comprise at least one peptide copper complex where the amino acid units of the peptide molecule thereof may be provided by amino acids other than alpha-amino acids. For example, the amino acids may be beta- or gamma-amino acids, such as those shown below.

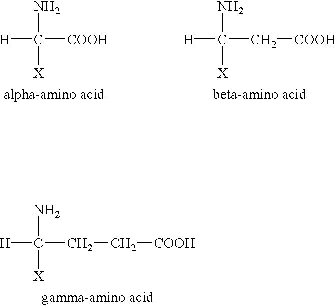

where X is the amino acid side-chain moiety.

Naturally occurring amino acids, that is, amino acids from which the amino acid units of naturally occurring proteins are derived, and their respective naturally occurring, amino acid side chain moieties, are shown below in Table 1. These naturally occurring amino acids are all in the L configuration, referring to the optical orientation of the alpha carbon or other carbon atom bearing the amino acid side chain. A peptide molecule may also comprise amino acids that are in the D optical configuration.

TABLE 1
NATURALLY OCCURRING AMINO ACID SIDE-CHAIN
MOIETIES

| Amino Acid Side Chain Moiety | Amino Acid |
|---|---|
| —H | Glycine |
| —CH₃ | Alanine |
| —CH(CH₃)₂ | Valine |
| —CH₂CH(CH₃)₂ | Leucine |
| —CH(CH₃)CH₂CH₃ | Isoleucine |
| —(CH₂)₄NH₃⁺ | Lysine |
| —(CH₂)₃NHC(NH₂)NH₂⁺ | Arginine |
| 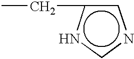 | Histidine |
| —CH₂COO— | Aspartic Acid |
| —CH₂CH₂COO— | Glutamic Acid |
| —CH₂CONH₂ | Asparagine |
| —CH₂CH₂CONH₂ | Glutamine |
| 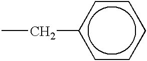 | Phenylalanine |
| 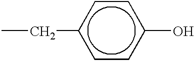 | Tyrosine |
| 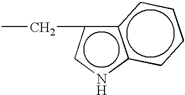 | Tryptophan |
| —CH₂SH | Cysteine |
| —CH₂CH₂SCH₃ | Methionine |
| —CH₂OH | Serine |
| —CH(OH)CH₃ | Threonine |

One example of a copper peptide complex is alanyl-histidyl-lysine:copper(II). Copper(II), as is well understood by the skilled artisan, designates a copper ion having a valence of 2 (e.g., Cu⁺²). Additional examples of the peptide copper complexes, encompassed in embodiments of the present invention, include, but are not limited to, those described in U.S. Pat. Nos. 4,665,054; 4,760,051; 4,767,753; 4,877,770; 5,023,237; 5,059,588; 5,120,831; 5,135,913; 5,145,838; 5,177,061; 5,214,032; 5,348,943; 5,538,945 and 5,550,183, incorporated herein by reference in their entireties.

In certain specific embodiments, the composition of the present invention comprises at least one peptide copper complex that is alanyl-histidyl-lysine:copper(II) (“AHK-Cu”), valyl-histidyl-lysine:copper(II) (“VHK-Cu”), or glycyl-histidyl-lysine:copper(II) (GHK-Cu″). Such peptides may be in either the L or D form. In a related, more specific embodiment, they are all in the L form.

Further, the expression “peptide copper complex,” as used herein, encompasses peptide copper complex derivatives. The expression “peptide copper complex derivative,” as used herein, refers to a peptide copper complex where the peptide molecule thereof has: 1) at least one amino acid side chain moiety that is a modification and/or variation of a naturally occurring, amino acid side-chain moiety; and/or 2) at least one of the hydrogens, bonded to an amide linkage nitrogen atom, substituted with a different moiety; and/or 3) the carboxyl group of the carboxyl terminal residue esterified or otherwise modified; and/or 4) at least one hydrogen, bonded to the nitrogen atom of the amino-terminal residue, substituted with a different moiety.

The amino acid side-chain moieties of the peptide copper complex derivatives may include alkyl, aryl, arylalkyl, alkoxy, or aryloxy moieties. As used herein, “alkyl” means a straight chain or branched, cyclic or noncyclic, substituted or unsubstituted, saturated or unsaturated aliphatic hydrocarbon containing from 1 to 18 carbon atoms. Representative saturated straight chain alkyls include methyl, ethyl, n-propyl and the like; while saturated branched alkyls include isopropyl, sec-butyl, isobutyl, tert-butyl, isopentyl, and the like. Representative, saturated cyclic alkyls include cyclopropyl, cyclobutyl, cyclopentyl, —CH₂cyclohexyl, and the like; while unsaturated cyclic alkyls include cyclopentenyl, cyclohexenyl, and the like. Unsaturated alkyls contain at least one double or triple bond between adjacent carbon atoms (referred to as an “alkenyl” or “alkynyl,” respectively). Representative alkenyls include ethylenyl, 1-butenyl, isobutylenyl, 2-methyl-2-butenyl, and the like; while representative alkynyls include acetylenyl, 2-butynyl, 3-methyl-1-butynyl, and the like.

Also, as used herein, “aryl” means an aromatic carbocyclic moiety such as phenyl or naphthyl, and may be substituted or unsubstituted. “Arylalkyl,” as used herein, means an alkyl having at least one alkyl hydrogen atom replaced with a substituted or unsubstituted aryl moiety, such as benzyl (i.e., —CH₂phenyl, —(CH₂)₂phenyl, —(CH₂)₃phenyl, —CH(phenyl)₂, and the like). As some examples, the amino acid side-chain moieties of alanine, valine, leucine, isoleucine and phenylalanine may generally be classified as alkyl, aryl or arylalkyl moieties.

“Alkoxy” and “aryloxy,” as used herein, refer, respectively, to alky and aryl moieties, as defined above, but each further comprising an oxygen atom used to link the moiety to the amino acid.

Additionally, the peptide copper complex derivative may, for example, be N-alkylated at one or more peptide bonds; and/or its carboxyl terminus may be esterified, for example, with a methyl, ethyl, or benzyl group, or may be reduced to a hydroxy or aldehyde. Additionally, the peptide copper complex derivative may, for example, be N-alkylated, N-acylated or N-sulfonylated at the amino terminus with, for example, methyl, benzyl, acetyl, benzoyl, methanesulfonyl, or fluorenyloxycarbonyl moieties.

Examples of the peptide copper complex derivatives, encompassed in embodiments of the present invention, include, but are not limited to, those disclosed and described in the above-cited U.S. patents that are directed to peptide copper complexes, as well as those disclosed and described in the published PCT application having the international publication number WO 94/03482, incorporated herein by reference in its entirety.

As one specific example, the disclosed composition may comprises a peptide copper complex derivative that is a derivative of GHK-Cu having the general formula:

[glycyl-histidyl-lysine-R]:copper(II)

where R is an alkyl moiety containing from 1 to 18 carbon atoms, an aryl moiety containing from 6 to 12 carbon atoms, an arylalkyl moiety, an alkoxy moiety containing from 1 to 12 carbon atoms, or an aryloxy moiety containing from 6 to 12 carbon atoms. This derivative of GHK-Cu is further described in the above-cited U.S. patents that are directed to peptide copper complexes.

The above compositions may be prepared from aqueous solutions of peptide copper complexes. Such solutions are prepared by methods that are well known to those skilled in the art. For example, an amount of dried peptide copper complex suitable for a desired concentration is readily dissolved in water with mixing and gentle heating. An alternative method is to prepare a solution of the desired peptide, followed by the addition of a copper salt in the desired molar ratio to yield the desired solution of the peptide copper complex. Examples of copper salts that may be used are cupric chloride and cupric acetate. When aqueous solutions of peptide copper complexes are prepared, the solutions are neutralized, typically with NaOH. In various embodiments of the inventive skin care composition of the present invention, the concentration of the at least one peptide copper complex, by weight of the composition, ranges from about 0.01% to about 5%, from about 0.025% to about 1%, and from about 0.05% to about 0.5%, respectively. Also, the molar ratio of peptide to copper in the complex ranges from about 1:1 to about 3:1 in some embodiments, and from about 1:1 to about 2:1 in other embodiments.

The present invention, in another embodiment, is also directed to compositions formed by combining at least one peptide copper complex with retinol, at least one retinol derivative, or a mixture thereof, where the combined compounds are encapsulated in liposomes or microsponges to aid in the delivery of the peptide copper complex or to increase the stability of the composition. In yet another embodiment of such compositions, the combined compounds may be formulated in an instrument allowing the delivery of the compounds via iontophoresis.

As has been noted above, it has been surprisingly found that a synergistic interaction is exhibited between retinol or retinol derivatives and peptide copper complexes when these components are combined in the compositions of the present invention. Specifically, the ability of retinol or a retinol derivative to effect cellular proliferation and differentiation is increased when a peptide copper complex is present, and the effectiveness of a peptide copper complex in reducing the signs of photodamage or aging in the skin is enhanced when retinol or a retinol derivative is present.

An active drug substance may be combined with a disclosed composition to provide a pharmaceutical preparation for skin. Further, a disclosed composition may provide a cosmetic preparation for skin, useful for treating signs of photodamaged and aging skin and for enhancing the appearance of normal skin. Such preparations may be in any form suitable for topical application, including: a cream, a lotion, a gel and a solution. Some examples of such cosmetic preparations, useful for cleansing, protecting and treating skin are: creams for the face, hands, feet, or the entire body (i.e., day creams, night creams, make-up removal creams, and foundation creams); make-up removal formulations; protective or skin care body milks; skin care lotions, gels, or foams (such as cleansing or disinfecting lotions); bath compositions; deodorant compositions; and aftershave and preshave gels or lotions.

The disclosed compositions of the present invention and the preparations provided thereby, may also contain at least one active ingredient, in addition to the retinol, retinol derivative, or mixture thereof, and the at least one peptide copper complex. Active ingredients, as defined herein, are compounds that provide benefits to the skin and/or desirable properties to the cosmetic formulations. In one embodiment, the active ingredient is a sunscreen agent, a tanning agent, a skin conditioning agent, a skin protectant, an emollient, or a humectant.

The sunscreen agent is generally an active ingredient that can absorb, reflect, or scatter radiation in the UV range at wavelengths from 290 to 400 nanometers. Specific examples include benzophenone-3 (oxybenzone), benzophenone-4 (sulisobenzone), benzophenone-8 (dioxybenzone), butyl methoxydibenzoylmethane (Avobenzone), DEA-methoxycinnamate (diethanolamine methoxycinnamate), ethyl dihydroxypropyl PABA (ethyl 4-[bis(hydroxypropyl)] aminobenzoate), ethylhexyl dimethyl PABA (Padimate O), ethylhexyl methoxycinnamate (octyl methoxycinnamate), ethylhexyl salicylate (octyl salicylate), homosalate, menthyl anthranilate (Meradimate), octocrylene, PABA (aminobenzoic acid), phenylbenzimidazole sulfonic acid (Ensulizole), TEA-salicylate (trolamine salicylate), titanium dioxide, and zinc oxide. One skilled in the art will appreciate that other sunscreen agents may be used in the compositions and preparations of the present invention.

Generally, skin conditioning agents include substances that enhance the appearance of dry or damaged skin, as well as materials that adhere to the skin to reduce flaking, restore suppleness, and generally improve the appearance of skin. Representative examples of a skin conditioning agent that may be combined with a disclosed composition, or preparation provided thereby, include: acetyl cysteine, N-acetyl dihydrosphingosine, acrylates/behenyl acrylate/dimethicone acrylate copolymer, adenosine, adenosine cyclic phosphate, adensosine phosphate, adenosine triphosphate, alanine, albumen, algae extract, allantoin and deriviatives, aloe barbadensis extracts, aluminum PCA, amyloglucosidase, arbutin, arginine, azulene, bromelain, buttermilk powder, butylene glycol, caffeine, calcium gluconate, capsaicin, carbocysteine, carnosine, beta-carotene, casein, catalase, cephalins, ceramides, chamomilla recutita (matricaria) flower extract, cholecalciferol, cholesteryl esters, coco-betaine, coenzyme A, corn starch modified, crystallins, cycloethoxymethicone, cysteine DNA, cytochrome C, darutoside, dextran sulfate, dimethicone copolyols, dimethylsilanol hyaluronate, DNA, elastin, elastin amino acids, epidermal growth factor, ergocalciferol, ergosterol, ethylhexyl PCA, fibronectin, folic acid, gelatin, gliadin, beta-glucan, glucose, glycine, glycogen, glycolipids, glycoproteins, glycosaminoglycans, glycosphingolipids, horseradish peroxidase, hydrogenated proteins, hydrolyzed proteins, jojoba oil, keratin, keratin amino acids, and kinetin.

Other examples a skin conditioning agent are: lactoferrin, lanosterol, lauryl PCA, lecithin, linoleic acid, linolenic acid, lipase, lysine, lysozyme, malt extract, maltodextrin, melanin, methionine, mineral salts, niacin, niacinamide, oat amino acids, oryzanol, palmitoyl hydrolyzed proteins, pancreatin, papain, PEG, pepsin, phospholipids, phytosterols, placental enzymes, placental lipids, pyridoxal 5-phosphate, quercetin, resorcinol acetate, riboflavin, RNA, saccharomyces lysate extract, silk amino acids, sphingolipids, stearamidopropyl betaine, stearyl palmitate, tocopherol, tocopheryl acetate, tocopheryl linoleate, ubiquinone, vitis vinifera (grape) seed oil, wheat amino acids, xanthan gum, and zinc gluconate. Skin conditioning agents other than those listed above may be combined with a disclosed composition or preparation provided thereby, as can be readily appreciated by one skilled in the art.

A skin protectant, for purposes of the present invention, refers to a compound that protects injured or exposed skin or mucous membrane surfaces from harmful or irritating external compounds. Representative examples thereof include: algae extract, allantoin, aluminum hydroxide, aluminum sulfate, betaine, camellia sinensis leaf extract, cerebrosides, dimethicone, glucuronolactone, glycerin, kaolin, lanolin, malt extract, mineral oil, petrolatum, potassium gluconate, and talc. One skilled in the art will readily appreciate that skin protectants other than those listed above may also be combined with a disclosed composition of the present invention or preparation provided thereby.

As noted, one or more emollients may also be combined with a disclosed composition. For purposes of the present invention, an emollient refers to a cosmetic ingredient that can help skin maintain a soft, smooth, and pliable appearance. Such emollients are able to provide these benefits, largely owing to their ability to remain on the skin surface or in the stratum corneum to act as a lubricant and reduce flaking. Some examples of emollients, suitable for embodiments of this invention, are: acetyl arginine, acetylated lanolin, algae extract, apricot kernel oil PEG-6 esters, avocado oil PEG-11 esters, bis-PEG-4 dimethicone, butoxyethyl stearate, C₁₈-C₃₆ acid glycol ester, C₁₂-C₁₃ alkyl lactate, caprylyl glycol, cetyl esters, cetyl laurate, coconut oil PEG-10 esters, di-C₁₂-C₁₃ alkyl tartrate, diethyl sebacate, dihydrocholesteryl butyrate, dimethiconol, dimyristyl tartrate, disteareth-5 lauroyl glutamate, ethyl avocadate, ethylhexyl myristate, glyceryl isostearates, glyceryl oleate, hexyidecyl stearate, hexyl isostearate, hydrogenated palm glycerides, hydrogenated soy glycerides, hydrogenated tallow glycerides, hydroxypropyl bisisostearamide MEA, isostearyl neopentanoate, isostearyl palmitate, isotridecyl isononanoate, laureth-2 acetate, lauryl polyglyceryl-6 cetearyl glycol ether, methyl gluceth-20 benzoate, mineral oil, myreth-3 palmitate, octyldecanol, octyldodecanol, odontella aurita oil, 2-oleamido-1,3 octadecanediol, palm glycerides, PEG avocado glycerides, PEG castor oil, PEG-22/dodecyl glycol copolymer, PEG shorea butter glycerides, phytol, raffinose, stearyl citrate, sunflower seed oil glycerides, and tocopheryl glucoside. One skilled in the art will readily appreciate that other emollients may also be used in embodiments of the skin care compositions, and related pharmaceutical and cosmetic preparations of this invention.

Humectants included in the above-mentioned embodiment of the present invention are cosmetic ingredients that help maintain moisture levels in skin. Some examples of suitable humectants are: acetyl arginine, algae extract, aloe barbadensis leaf extract, betaine, 2,3-butanediol, chitosan lauroyl glycinate, diglycereth-7 malate, diglycerin, diglycol guanidine succinate, erythritol, fructose, glucose, glycerin, honey, hydrolyzed wheat protein/PEG-20 acetate copolymer, hydroxypropyltrimonium hyaluronate, inositol, lactitol, maltitol, maltose, mannitol, mannose, methoxy PEG, myristamidobutyl guanidine acetate, polyglyceryl sorbitol, potassium PCA, propylene glycol, sodium PCA, sorbitol, sucrose, and urea. Other humectants may be used for embodiments of this invention, as will be appreciated by one skilled in the art.

In addition to the active ingredients described above, the disclosed compositions and preparations provided thereby may also contain inert, physiologically acceptable carriers or diluents. Suitable carriers or diluents include, but are not limited to: water, physiological saline, bacteriostatic saline (e.g.., saline containing 0.9 mg/ml benzyl alcohol), petrolatum based creams (e.g., USP hydrophilic ointments and similar creams), various types of pharmaceutically acceptable gels, and short chain alcohols and glycols (e.g., ethyl alcohol and propylene glycol).

In another embodiment, yet additional ingredients may be combined with the compositions of the present invention, including fatty alcohols, fatty acids, organic or inorganic bases, preserving agents, wax esters, steroid alcohols, triglyceride esters, phospholipids such as lecithin and cephalin, polyhydric alcohol esters, fatty alcohol ethers, hydrophilic lanolin derivatives, hydrophilic beeswax derivatives, cocoa butter waxes, silicon oils, pH balancers, cellulose derivatives, and hydrocarbon oils such as palm oil, coconut oil, and mineral oil. Additional ingredients that are particularly useful, as is well understood by those skilled in the art, are those that may be used to vary the texture, viscosity, color and appearance of the dislcosed compositions and preparations, and include emulsifying agents, thickening agents, and surfactants.

Emulsifiers and surfactants are used in preparing embodiments of the present invention directed to compositions and preparations formulated as emulsions. Either water in oil or oil in water emulsions may be formulated. Examples of suitable surfactants and emulsifying agents include: nonionic ethoxylated and nonethoxylated surfactants, abietic acid, almond oil PEG, beeswax, butylglucoside caprate, C₁₈-C₃₆ acid glycol ester, C₉-C₁₅ alkyl phosphate, caprylic/capric triglyceride PEG-4 esters, ceteareth-7, cetyl alcohol, cetyl phosphate, corn oil PEG esters, DEA-cetyl phosphate, dextrin laurate, dilaureth-7 citrate, dimyristyl phosphate, glycereth-17 cocoate, glyceryl erucate, glyceryl laurate, hydrogenated castor oil PEG esters, isosteareth-11 carboxylic acid, lecithin, lysolecithin, nonoxynol-9, octyldodeceth-20, palm glyceride, PEG diisostearate, PEG stearamine, poloxamines, polyglyceryls, potassium linoleate, PPG's, raffinose myristate, sodium caproyl lactylate, sodium caprylate, sodium cocoate, sodium isostearate, sodium tocopheryl phosphate, steareths, TEA-C₁₂-C₁₃ pareth-3 sulfate, tri-C₁₂-C₁₅ pareth-6 phosphate, and trideceths. Other surfactants and emulsifiers may be used, as will be appreciated by one skilled in the art.

Further, disclosed compositions and preparations provided thereby may also include thickening or viscosity increasing agents. Suitable examples include those agents commonly used in skin care preparations, such as: acrylamides copolymer, agarose, amylopectin, bentonite, calcium alginate, calcium carboxymethyl cellulose, carbomer, carboxymethyl chitin, cellulose gum, dextrin, gelatin, hydrogenated tallow, hydroxytheylcellulose, hydroxypropylcellulose, hydroxpropyl starch, magnesium alginate, methylcellulose, microcrystalline cellulose, pectin, various PEG's, polyacrylic acid, polymethacrylic acid, polyvinyl alcohol, various PPG's, sodium acrylates copolymer, sodium carrageenan, xanthan gum, and yeast beta-glucan. Thickening agents other than those listed above may also be used in embodiments of this invention.

Excipients may also be combined with disclosed compositions and preparations. A suitable excipient is adapted for application to the face and neck. More specifically, a suitable excipient should have a high affinity for the skin, be well tolerated, stable, and yield a consistency that allows for easy and pleasant utilization.

The compositions of the present invention, as well as the pharmaceutical and cosmetic preparations for skin provided thereby, are intended primarily as products for topical application to human skin. Accordingly, in one embodiment the disclosed composition is in the form of a cream, gel, fluid cream or milk, lotion, or oil.

A further aspect of this invention is directed to a method for treating skin to condition and smoothen the skin, lessen hyperpigmentation, and prevent or reduce the appearance of wrinkles and signs of photodamage and aging of the skin. The method comprises contacting the skin in need thereof with an effective amount of a disclosed composition. As a more specific example, a small amount of material (from about 1 to about 5 ml) is applied to exposed areas of skin in need of treatment from a suitable container or applicator, and, if necessary, the material is then spread over and/or rubbed into the skin using the hand or finger, or a suitable device. Each of the compositions and preparations disclosed herein is typically packaged in a container to suit its viscosity and intended use by the consumer. For example, a lotion or fluid cream may be packaged in a bottle, roll-ball applicator, capsule, propellant-driven aerosol device, or a container fitted with a manually operated pump. A cream can simply be stored in a non-deformable bottle or squeeze container, such as a tube or a lidded jar.

The following examples are provided for the purpose of illustration, not limitation.

### EXAMPLES

The examples which follow illustrate the preparation, characterization and utility of certain exemplary embodiments of the present invention.

### Example 1

### A Moisturizing Lotion Incorporating the Composition of this Invention

| Ingredients | % w/w |
|---|---|
| Water | 73.80% |
| Glycerin | 1.00% |
| xanthan gum | 0.50% |
| diisopropyl adipate | 4.00% |
| isocetyl stearate | 6.00% |
| octyl palmitate | 10.00% |
| glyceryl stearate | 1.00% |
| cetyl alcohol | 1.00% |
| stearyl alcohol | 0.80% |
| behenyl alcohol | 0.50% |
| palmitic acid | 0.25% |
| stearic acid | 0.25% |
| glycyl-L-histidyl-L-lysine copper complex | 0.20% |
| Retinol | 0.10% |
| propylene glycol | 0.55% |
| diazolidinyl urea | 0.03% |
| iodopropynyl butylcarbonate | 0.02% |
| total | 100.00% |

### Example 2

### A Moisturizing Cream Incorporating the Composition of this Invention

| Ingredients | % w/w |
|---|---|
| purified water | 76.35% |
| ethylhexyl palmitate | 8.00% |
| Octyldodecanol | 2.50% |
| butyloctyl calicylate | 2.00% |
| Squalane | 1.50% |
| jojoba oil | 0.50% |
| tocopheryl acetate | 0.20% |
| Bisabolol | 0.20% |
| Polyacrylamide | 1.50% |
| laureth-7 | 0.50% |
| Glycerin | 3.00% |
| Panthenol | 0.60% |
| Allantion | 0.10% |
| Cyclomethicone | 0.50% |
| Carbomer | 0.10% |
| polysorbate 20 | 0.20% |
| glycyl-L-histidyl-L-lysine copper | 0.25% |
| complex | |
| Retinol | 1.00% |
| propylene glycol | 0.56% |
| diazolidinyl urea | 0.30% |
| Methylparaben | 0.11% |
| Propylparaben | 0.03% |
| total | 100.00% |

### Example 3

### A Body Lotion Incorporating the Composition of this Invention

| Ingredients | % w/w |
|---|---|
| Water | 74.35% |
| hydrogenated vegetable oil | 6.00% |
| canola oil | 4.00% |
| polyoxyethylene stearyl stearate | 4.00% |
| steareth-21 | 2.00% |
| Octyldodecanol | 6.00% |
| sorbeth-30 | 2.50% |
| glycyl-L-histidyl-L-lysine copper complex | 0.10% |
| retinol palmitate | 0.05% |
| propylene glycol | 0.56% |
| diazolidinyl urea | 0.30% |
| Methylparaben | 0.11% |
| Propylparaben | 0.03% |
| Total | 100.00% |

### Example 4

### A Water-In-Oil Emulsion Incorporating the Composition of this Invention

| Ingredients+ | % w/w |
|---|---|
| purified water | 73.49% |
| quarternium 82 | 2.00% |
| polyquarternium-37 | 1.10% |
| mineral oil | 0.50% |
| PPG-1-trideceth-6 | 0.40% |
| ethylhexyl isononanoate | 20.00% |
| cetyl dimethicone copolyol | 1.00% |
| glycyl-L-histidyl-L-lysine copper complex | 0.10% |
| retinyl acetate | 0.30% |
| Retinol | 0.05% |
| propylene glycol | 0.56% |
| imidazolidinyl urea | 0.30% |
| Methylparaben | 0.11% |
| Propylparaben | 0.03% |
| Butylparaben | 0.02% |
| Isopropylparaben | 0.02% |
| Isobutylparaben | 0.02% |
| Total | 100.00% |

### Example 5

### An Oil-In-Water Emulsion Type Face Cream Incorporating the Composition of this Invention

| Ingredients | % w/w |
|---|---|
| purified water | 75.20% |
| Glycerin | 4.00% |
| steareth-100 | 0.50% |
| steareth-2 | 0.25% |
| xanthan gum | 0.25% |
| isopropyl palmitate | 4.00% |
| Isohexanodecane | 1.00% |
| isostearyl isostearate | 1.20% |
| octyl dodecanol | 1.00% |
| stearic acid | 2.50% |
| cetostearyl alcohol | 2.50% |
| Petrolatum | 4.00% |
| glycyl-L-histidyl-L-lysine copper | 0.10% |
| complex | |
| retinyl palmitate | 0.30% |
| Phenoxyethanol | 3.00% |
| Methylparaben | 0.11% |
| Propylparaben | 0.03% |
| Butylparaben | 0.02% |
| Isopropylparaben | 0.02% |
| Isobutylparaben | 0.02% |
| Total | 100.00% |

### Example 6

### A High Silicon Content Cream Incorporating the Composition of this Invention

| Ingredients | % w/w |
|---|---|
| purified water | 43.25% |
| Dimethicone | 50.00% |
| behentriomnium methosulfate | 4.00% |
| cetearyl alcohol | 2.00% |
| glycyl-L-histidyl-L-lysine copper | 0.20% |
| complex | |
| retinyl palmitate | 0.10% |
| Methylparaben | 0.30% |
| Ethylparaben | 0.10% |
| Propylparaben | 0.03% |
| Butylparaben | 0.02% |
| Total | 100.00% |

### Example 7

The efficacy of the disclosed skin care compositions of this invention can be demonstrated via standard assays used to assess the performance of skin care products. For example, the compositions of this invention can be provided to volunteer subjects having signs of photo damaged skin such as age spots, hyperpigmentation, fine lines and wrinkles. These signs of clinical aging could be rated using, for example, a scale of 0-9 at baseline, and at weeks 4 and 8. Subjects could be given topical preparations, formulated according to the present invention, along with instructions that the topical preparations are to be applied twice daily to the areas showing signs of photodamage and aging. Clinical photographs may also be taken for comparison.

At the end of 4 and 8 weeks, the clinical signs of aging would again be assessed, and corresponding photographs taken for comparison with those taken earlier and subsequently. Comparison of data with the data collected earlier and subsequently would reveal a diminishment of the clinical signs of aging and photodamaged skin as a result of the treatment with the composition with the skin care compositions and preparations of this invention.

All of the above U.S. patents, U.S. patent application publications, U.S. patent applications, foreign patents, foreign patent applications and non-patent publications referred to in this specification and/or listed in the Application Data Sheet, are incorporated herein by reference, in their entirety.

From the foregoing, it will be appreciated that, although specific embodiments of the invention have been described herein for purposes of illustration, various modifications may be made without deviating from the spirit and scope of the invention. Accordingly, the invention is not limited except as by the appended claims.

1. A composition comprising retinol, a retinol derivative, or a mixture thereof, and a peptide copper complex.

2. The composition of claim 1 wherein the retinol is all-trans retinol, 1,3-cis-retinol, 3,4-didehydro-retinol, or 9-cis-retinol.

3. The composition of claim 1 wherein the retinol derivative is an ester of retinol.

4. The composition of claim 3 wherein the ester of retinol is a C₁-C₃₀ ester of retinol.

5. The composition of claim 3 wherein the ester of retinol is a C₂-C₂₀ ester of retinol.

6. The composition of claim 3 wherein the ester of retinol is a C₂, C₃ or C₁₆ ester of retinol.

7. The composition of claim 3 wherein the ester of retinol is retinyl palmitate, retinyl acetate or retinyl proprionate.

8. The composition of claim 1 wherein the retinol derivative is retinoic acid or retinyl aldehyde.

9. The composition of claim 1 wherein the retinol, the retinol derivative, or mixture thereof, is present at a concentration ranging from about 0.001% to about 10% by weight of the composition.

10. The composition of claim 1 wherein the retinol, the retinol derivative, or mixture thereof, is present at a concentration ranging from about 0.01% to about 1% by weight of the composition.

11. The composition of claim 1 wherein the retinol, the retinol derivative, or mixture thereof, is present at a concentration ranging from about 0.01% to about 0.5% by weight of the composition.

12. The composition of claim 1 wherein the peptide copper complex is L-alanyl-L-histidyl-L-lysine:copper(II).

13. The composition of claim 1 wherein the peptide copper complex is L-valyl-L-histidyl-L-lysine:copper(II).

14. The composition of claim 1 wherein the peptide copper complex is glycyl-L-histidyl-L-lysine: copper(II).

15. The composition of claim 1 wherein the molar ratio of peptide to copper in the peptide copper complex ranges from about 1:1 to about 3:1.

16. The composition of claim 1 wherein the molar ratio of peptide to copper in the peptide copper complex ranges from about 1:1 to about 2:1.

17. The composition of claim 1 wherein the peptide copper complex is present at a concentration ranging from about 0.01% to about 5% by weight of the composition.

18. The composition of claim 1 wherein the peptide copper complex is present at a concentration ranging from about 0.025% to about 1% by weight of the composition.

19. The composition of claim 1 wherein the concentration of the peptide copper complex is present at a concentration ranging from about 0.05% to about 0.5% by weight of the composition.

20. The composition of claim 1 wherein the retinol, the retinol derivative, or a mixture thereof, and the peptide copper complex is encapsulated in a liposome or microsponge adapted to aid in the delivery of the peptide copper complex, or to enhance the stability of the composition.

21. The composition of claim 1 wherein the retinol, the retinol derivative, or mixture thereof, and the peptide copper complex are formulated in an instrument adapted to deliver the compounds via iontophoresis.

22. The composition of claim 1, further comprising an inert and physiologically-acceptable carrier or diluent.

23. The composition of claim 22 wherein the inert and physiologically-acceptable carrier or diluent is water, physiological saline, bacteriostatic saline, a petrolatum based cream, a pharmaceutically acceptable gel, a short chain alcohol, or a short chain glycol.

24. The composition of claim 1, further comprising a sunscreen agent, a skin conditioning agent, a tanning agent, a skin protectant, an emollient, or a humectant.

25. The composition of claim 1, further comprising a fatty alcohol, a fatty acid, an organic base, an inorganic base, a preserving agent, a wax ester, a steroid alcohol, a triglyceride ester, a phospholipid, a polyhydric alcohol ester, a fatty alcohol ether, a hydrophilic lanolin derivative, a hydrophilic beeswax derivative, a cocoa butter wax, a silicon oil, a pH balancer, a cellulose derivative, a hydrocarbon oil, or a mixture thereof.

26. The composition of claim 1, further comprising an emulsifying agent, a surfactant, a thickening agent, an excipient, or a mixture thereof.

27. The composition of claim 1 wherein the composition is in the form of a solution, cream, gel, fluid cream, lotion, or oil.

28. A method for cosmetically treating skin, comprising contacting an area of the skin in need thereof with an effective amount of the composition of claim 1.

29. The method of claim 28 wherein the cosmetic treatment of skin is smoothening the skin, reducing hyperpigmentation of the skin, reducing wrinkles in the skin, reducing evidence of photodamage of the skin, or reducing the signs of aging in the skin.
